(19) United States
(12) Patent Application Publication
(10) Pub. No.: US 2022/0383996 A1
(43) Pub. Date: Dec. 1, 2022

(54) ASSIGNING PEPTIDES TO PEPTIDE GROUPS FOR VACCINE DEVELOPMENT

(71) Applicant: Amazon Technologies, Inc., Seattle, WA (US)

(72) Inventors: Layne Christopher Price, Seattle, WA (US); David Heckerman, Bellevue, WA (US); Frank Wilhelm Schmitz, Seattle, WA (US)

(21) Appl. No.: 17/332,719

(22) Filed: May 27, 2021

(51) Int. Cl. G16H 10/60; A61K 39/00; G16B 15/20; G16H 50/20

(52) U.S. Cl. CPC G16H 10/60; A61K 39/0011; G16B 15/20; G16H 50/20

(57) ABSTRACT

Techniques are described and relate to assigning peptides to peptide groups for vaccine development. In an example, a peptide property of a peptide is determined, where this peptide is from different peptides that are to be assigned to different groups of vaccine. A determination is also made that the peptide is to be assigned to a first group from the different groups based at least in part on the peptide property. The first group has a first group property that is based at least in part on peptide properties of first peptides to be assigned to the first group. The first group property is within a similarity range relative to a second group property of a second group from the different groups. Information is generated and indicates that the peptide is assigned to the first group.

### BACKGROUND

Various applications are available in the life sciences space based on major histocompatibility complex (MEW) molecules and peptides that are bound by MEW molecules. For instance, an understanding can be developed about the functions of an immune system, such as the interactions between T-cells and antigen-presenting cells. This understanding can be used for diagnosis and disease identification, drug discovery, and vaccine development.

### BRIEF DESCRIPTION OF THE DRAWINGS

Various embodiments in accordance with the present disclosure will be described with reference to the drawings, in which:

FIG. 1 illustrates an example of a cancer vaccine that includes multiple solutions corresponding to different peptide groups according to embodiments of the present disclosure;

FIG. 2 illustrates an example of assigning peptides to peptide groups for cancer vaccine according to embodiments of the present disclosure;

FIG. 3 illustrates an example of a computing environment for defining peptide groups for a cancer vaccine according to embodiments of the present disclosure;

FIG. 4 illustrates an example of a flow for defining peptide groups for a cancer vaccine according to embodiments of the present disclosure;

FIG. 5 illustrates an example of a flow for assigning peptides to peptide groups for cancer vaccine according to embodiments of the present disclosure;

FIG. 6 illustrates an example of sorting peptides according to embodiments of the present disclosure;

FIG. 7 illustrates an example of defining tiers according to embodiments of the present disclosure;

FIG. 8 illustrates an example of a tier-based assignment of peptides to peptide groups according to embodiments of the present disclosure;

FIG. 9 illustrates an example of tier-based shuffling according to embodiments of the present disclosure;

FIG. 10 illustrates an example of redefining tiers according to embodiments of the present disclosure; and

FIG. 11 illustrates aspects of an example environment for implementing aspects in accordance with various embodiments.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Embodiments of the present disclosure relate to, among other things, assigning peptides to peptide groups for vaccine development. In an example, multiple peptides are identified for a subject as candidate peptides for a vaccine personalized for the subject, where these peptides are predicted to trigger a positive immunogenic response in the subject (e.g., a CD8+ immunogenic response and/or a CD4+ immunogenic response) based on their binding with major histocompatibility complex (MHC) molecules of the subject (e.g., MHC class I molecules and/or MHC class II molecules). The peptides can be assigned to peptide groups, each group used in a vaccine composition, where each vaccine composition can be administered (e.g., injected in a same or a different location of the subject). In particular, a peptide group identifies a subset of the candidate peptides for a vaccine composition. These identified peptides are formulated for administration, for example with an adjuvant, immunostimulant or both. For example in one embodiment, the identified peptides are combined in a solution (which optionally can include an adjuvant, immunostimulant or both, e.g., polyinosinic-polycytidylic acid (Poly ICLC), and a solvent, e.g., dimethyl sulfoxide (DMSO) at particular concentrations) to form a vaccine composition. The assignment of peptides to the different peptide groups is performed in a manner in which the resulting peptide groups have a similar immunogenicity response (e.g., a response that can be determined to have a score within a predefined score range). For peptides assigned to a peptide group, the peptides are used in the manufacturing of the corresponding vaccine composition. In this way, different vaccine compositions can be expected to trigger the immunogenicity response with a similar efficacy. Optionally, peptide co-solubility is also determined and peptide groups that are co-soluble are selected, thereby ensuring co-soluble peptides in the vaccine composition.

To illustrate, consider an example of a cancer vaccine to be developed for a human patient exhibiting a particular type of cancer. A biopsy can be performed on the human patient (e.g., on healthy or cancerous cells thereof) and genome sequencing can be applied to the biopsy to determine MHC class II alleles (for example for human leukocyte antigen (HLA)-DP, HLA-DQ, and HLA-DR) expressed in the human patient. An artificial intelligence model can be used to identify a number (for example, sixty) neo-antigen peptides, each having a likelihood for triggering a positive immunogenic response (e.g., a CD4+ immunogenic response) by binding, in the human patient's body, with MHC class II molecules. For a peptide, the corresponding likelihood represents a class II immunogenicity score. A peptide manufacturer can indicate those peptides that can be manufactured based on solubility or other criteria (for example, that forty out of the sixty neo-antigen peptides can be manufactured). Neo-antigen peptides (e.g., forty of them) identified for manufacture are usable for developing a cancer vaccine of the human patient. Out of these neo-antigen peptides, a subset (e.g., eighteen) of them can be selected as candidate peptides. Optionally additional peptides, such as “universal” (that bind MHC molecules from various alleles) class II peptides can be added to the peptides under consideration. Exemplary universal peptides can include but are not limited to the “PADRE” peptide (Smahel et al, Gene Therapy volume 21, pages 225-232(2014)). For example, two PADRE peptides can be included for a total of twenty peptides. In a first vaccine plan, the twenty peptides are assigned to four peptide groups, each including five peptides from the different twenty peptides. The four peptides groups can be completely different (e.g., they do not contain any duplicate peptides) or some amount of overlap may be allowed (e.g., no more than one duplicated peptide). Further, the average class II immunogenicity scores of the different peptide groups are within a similarity range (e.g., within a plus/minus five or ten or twenty percent relative range). One or more additional vaccine plans can be further defined from the same twenty peptides by using another assignment(s) of the twenty peptides to four peptide groups. Additionally or alternatively, one or more additional vaccine plans can be defined by substituting, before the assignment(s), one or more of the eighteen neo-antigen peptides with one or more of the remaining twenty-two neo-antigen peptides out of the initial candidate set of forty. Like in the first vaccine plan, the different peptide groups within each additional vaccination plan have a similar average class II immunogenicity score. The different vaccine plans can be identified to the peptide manufacturer. Upon confirmation of the peptide manufacturer that, a vaccine plan has peptide groups that contain co-soluble peptides, the manufacturing of four compositions of the cancer vaccine is triggered using the vaccine plan. Each composition corresponds to a different peptide group of the vaccine plan and comprises the peptides assigned to the peptide group.

Embodiments of the present disclosure provide several advantages. For example, a personalized vaccine can be developed for a subject. In embodiments in which a vaccine comprises multiple compositions, each composition can contain multiple co-soluble peptides. Further, the immunogenicity responses of the compositions will be similar such that the compositions can be expected to trigger an immunogenicity response at a similar efficacy when injected at different sites of the subject.

In the interest of clarity of explanation, various embodiments are described herein using examples of peptides for cancer vaccine of a human patient. However, the embodiments are not limited to such examples. For example, the embodiments similarly apply to other types of peptide-based vaccines targeted for a particular immune response and to other types of subject (e.g., mammals or other animals). Generally, candidate peptides can be identified for the subject based on, for instance, a prediction of a targeted immunogenicity response. For cancer vaccine, these peptides include neo-antigen peptides. For other types of vaccines, other types of peptides can be used where a peptide type(s) can depend on the target immune response. The peptides can be assigned to different peptide groups of vaccine compositions, where the immunogenicity properties of the different peptide groups are similar. An immunogenicity property of a peptide group can be defined based on biological and/or chemical properties of individual peptides or of a collection of peptides that are assigned to the peptide group. In the case of cancer vaccine, this property can include, for instance, a class I immunogenicity score, a class II immunogenicity score, an amino acid sequence length measure, the distribution or frequency of cysteine amino acids in the peptides of the peptide group.

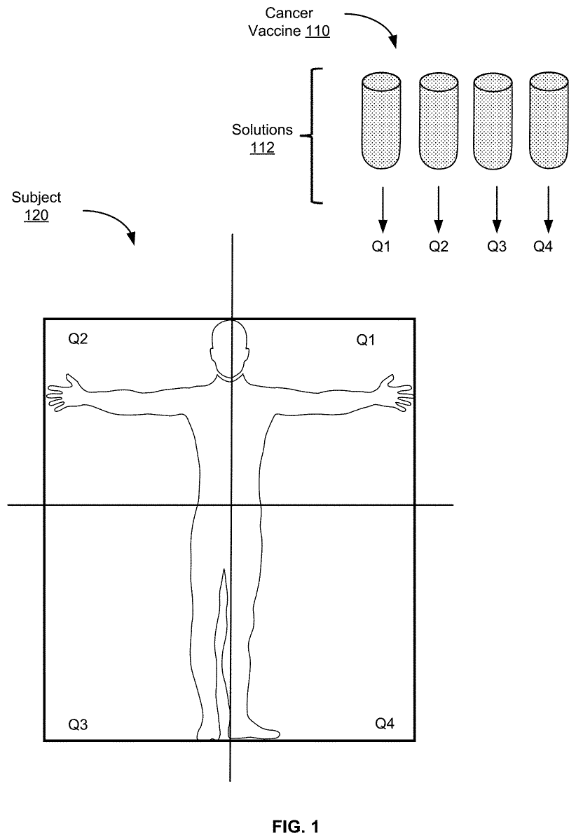

FIG. 1 illustrates an example of a cancer vaccine 110 that includes multiple solutions 112 corresponding to different peptide groups according to embodiments of the present disclosure. The cancer vaccine 110 can be personalized to a specific subject 120, where each of the solutions 112 contains certain peptides predicted to trigger a positive immunogenic response by binding with MHC molecules of the subject 120.

In an example, the cancer vaccine 110 includes four solutions 112 (or some other total number of solutions 112). In turn, each of the solutions 112 includes five peptides that differ by amino acid sequence from each other (or some other total number of peptide types)), where peptides are added to the solution at a particular concentration. The peptides can vary by at least one peptide between the solutions 112. It is also possible that the solutions 112 (i.e., different solutions) do not contain duplicate neo-antigen peptides. However, in some embodiments, a subset of the solutions 112 (e.g., one or two of them) can contain duplicate universal peptides. A universal peptide (also referred to in the literature as a “promiscuous peptide”) can be, in an example, a peptide capable of binding to at least a majority of commonly found HLA-DR alleles, and in some embodiments also to HLA-DQ and HLA-DP alleles. See, e.g., Sinigaglia, et al., Current Opinion in Immunology 6(1): February 1994, Pages 52-56. Universal peptides can therefore be applicable to a population of subject. Exemplary universal peptides can include but are not limited to the “PADRE” peptide (Smahel et al, Gene Therapy volume 21, pages 225-232(2014)), or pan-DR binding peptides (see, e.g., U.S. Pat. No. 9,249,187) or diphtheria or tetanus toxoid (TT), for example TT₈₃₀₋₈₄₄ or TpD (Fraser, et al., Vaccine 32(24), 19 May 2014, Pages 2896-2903).

The concentration of the peptides in a composition (or, the total number of peptides across compositions) can be the same or can vary within a predefined concentration range. For instance, in some embodiments, the solution contains 0.3 milligram per millimeter (mg/mL) of each peptide type for a total concentration of 1.5 mg/mL of peptides. The solution can also include other ingredients at corresponding concentrations, such as NaCl at 0.9 percent or 0.5 mg/mL Poly ICLC and 4% DMSO. Of course, other concentrations are possible for any of the peptides and/or ingredients and depend on the type of the subject 120 and applicable regulatory requirements (e.g., a concentration of up to twenty percent DMSO can be used for a human patient, whereas this concentration can be higher for another type of mammal, such as up to seventy percent).

The four solutions 112 can be formulated, for example, for injection in the subject 120. The injection can be at a same location (e.g., the left arm). Alternatively, each of the four solutions 112 can be injected at a different location of the subject 120. For instance, four quadrants are identified on the subject 120 (illustrated in FIG. 1 as “Q1,” “Q2,” “Q3,” and “Q4”). Each of the four solutions 112 is associated with one of the quadrants and can be injected at a location within the quadrant (e.g., left arm, right arm, right leg, or left leg). The associations can be predefined, as further described herein below.

Although FIG. 1 illustrates that the cancer vaccine 110 uses a liquid form, other cancer vaccine types are possible. For instance, the cancer vaccine 110 can be similarly developed to include multiple pills (e.g., four pills) or sprays, each containing a different set of peptide types (e.g., five peptides).

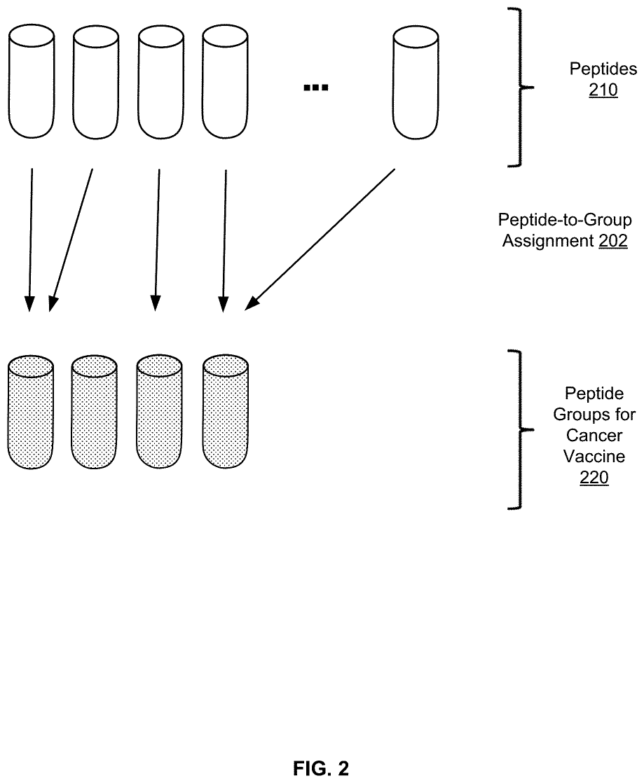

FIG. 2 illustrates an example of assigning 202 peptides 210 to peptide groups 220 for cancer vaccine according to embodiments of the present disclosure. The peptide groups 220 can be used to define solutions, each representing a peptide pool. Upon determining that the peptides in a peptide pool are co-soluble, the peptide pool can be used as a vaccine shot.

In an example, peptides are identified for a subject as candidate peptides expected to trigger a targeted immunogenic response of the subject based on the MHC molecules of the subject. Solubility of each peptide is tested. The peptides found to be soluble are identified as part of the peptides 210. Subsequently, a peptide-to-group assignment 202 is performed on the peptides 210 to distribute the peptides 210 into a peptide groups 220 for cancer vaccine. The different peptide groups 220 have a similar immunogenicity properties. If the peptides within each peptide group are found to be co-soluble, these peptide groups 220 are identified in a vaccine plan for the subject and can be manufactured into vaccine shots.

Identifying the peptides can rely on an artificial intelligence model trained to output identifiers of these peptides (e.g., a sequence of amino acids defining each peptide) based on information about MHC alleles of the subject. To illustrate, consider an example of a human patient. The human patient may have six types of MHC class II molecules (e.g., MHC alleles), referred to as HLA class II molecules. By using genome sequencing (e.g., next generating sequencing (NGS) on a biopsy from the human patient, a set (e.g., a set of six or eight) of HLA class II molecules of the human patient is determined (e.g., each element in the set identifies one HLA allele). A database of neo-antigen peptides can be available for development of a cancer vaccine. Data identifying the HLA class II molecules can be input to the artificial model. This model pairs neo-antigen peptides from the database with the HLA class II molecules of the subject to generate candidate pairs of peptide-HLA class II molecules. For each candidate pair, the artificial intelligence model generates a CD4+ immunogenic response prediction indicating the likelihood that the candidate pair would elicit a positive CD4+ immunogenic response. The candidate pairs with the highest likelihoods (or likelihoods exceeding a predefined threshold) are selected and ranked (e.g., in a descending order of their CD4+ immunogenic response prediction likelihood).

Out of the above ranked neo-antigen peptides, a total number of neo-antigen peptides are selected. For instance, the sixty top ranked neo-antigen peptides (or some other number) are selected. These peptides are identified to a peptide manufacturer by, for instance, identifying thereto the amino acid sequence of each neo-antigen peptide. The term “neo-antigen” refers to cancer antigens.

A peptide manufacturer, which may but need not be different from an entity that is responsible for generating peptide groups, can test the manufacturability and solubility of each neo-antigen peptide. For instance, the peptide manufacturer generates, if possible, an amount of a neo-antigen peptide given an identified amino acid sequence, and adds this amount to a solution at a particular concentration (e.g., 1.5 mg/mL). The solution includes other ingredients at other concentrations, such as sodium chloride (NaCl) at 0.9 percent and DMSO at four percent, with a pH between six and eight. Other values of the concentrations can be used (e.g., between one and three mg/mL for the neo-antigen peptide, 0.5 to 1.5 percent for NaCl, and two to twenty percent for DMSO). If this manufactured peptide is found to be soluble, the peptide manufacturer returns information indicating so. Otherwise, the peptide manufacturer indicates that the neo-antigen peptide cannot be manufactured or is not soluble, as applicable. As such, out of the initial set of sixty neo-antigen peptides, the peptide manufacturer identifies a subset (e.g., forty or some other number) of possible neo-antigen peptides.

Out of the forty (or some other number) of neo-antigen peptides, a smaller subset of neo-antigen peptides (e.g., eighteen of them) is selected and corresponds to the peptides 210. For instance, this subset corresponds to the eighteen top ranked neo-antigen peptides. A number (e.g., one or two) of PADRE peptides (and/or other types of universal peptides) are also identified and are included in the peptides 210, for a total of twenty peptides. In this illustration, the peptides 210 form a set that has twenty elements, although a different size of the set is possible. This set is to be distributed into four peptide groups 220 (or some other number), each possible to be used in a different solution of a cancer vaccine. Thus, each peptide group 220 can include five peptides (or some other number that depends on the size of the set and the number of the to be developed solutions).

Next, the peptide-to-group assignment 202 is performed using one or more assignment techniques to assign the twenty peptides into the four peptide groups 210. Generally, these techniques ensure that the immunogenicity response of the peptide groups 220 are similar, while also the peptide groups 220 themselves are different (e.g., each peptide group differs from the remaining peptide groups by at least by one peptide, more than one peptide or all neo-antigen peptides between groups are different). The techniques can be categorized in two categories. In a first category, random assignments of the peptides 210 into peptide groups are generated and then the overall immunogenicity response of the resulting peptide groups are estimated. If these immunogenicity responses are similar, the peptide groups are set as the peptide groups 220. Otherwise, another random assignment is performed. In a second category, the assignment itself ensures that the resulting peptide groups have similar immunogenicity responses without the need to estimate the immunogenicity responses after the assignment. An example of such techniques include a combinational optimization algorithm and a tournament-style algorithm. These techniques are further described herein below.

The techniques above can also be subject to different assignment rules. An assignment rule can be a filtering rule. For instance, if a peptide group includes a peptide with more than one cysteine in its sequence, the filtering rule can remove the peptide group. An assignment rule can also be a duplication rule. For instance, the duplication rule can specify that only two of the peptide groups 220 can include a PADRE peptide. The duplication rule can also specify that no neo-antigen peptide duplication is allowed, or if one is allowed, the maximum number of allowed neo-antigen peptide duplications.

Regardless of the assignment techniques, and upon the application of the assignment rule(s), the peptide-to-group assignment 202 results in the definition of four peptide groups 220 (or, the relevant number) per vaccine plan. These peptide groups 220 are identified to the peptide manufacturer by, for instance, identifying the vaccine plan, a label of the peptide group, and the amino acid sequence of each peptide per peptide group.

Here, the peptide manufacturer can test the co-solubility of the peptides per peptide group. For instance, per peptide group of a vaccine plan, the peptide manufacturer adds amounts of the identified peptides to a solution (e.g., 0.3 mg/ML per peptide into a solution of 0.9 percent NaCl and four percent DMSO at a pH between six and eight, although other concentrations are possible as described herein above) to then perform a co-solubility test. If each peptide group of a vaccine plan contains co-soluble peptides, the peptide manufacturer returns information indicating so. Otherwise, the peptide manufacturer indicates that the vaccine plan is not proper and can identify the particular culpable peptide group(s).

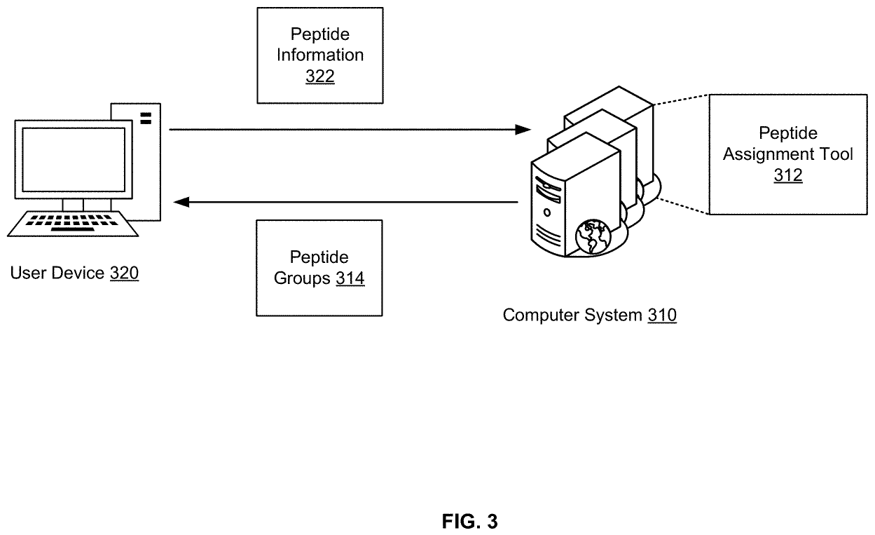

FIG. 3 illustrates an example of a computing environment for defining peptide groups for a cancer vaccine according to embodiments of the present disclosure. The computing environment includes a computer system 310 that hosts a peptide assignment tool 312. The computing environment also includes a user device 320 that is communicatively coupled with the computer system 310 over a data network (e.g., the Internet). The user device 320 can send peptide information 322 about a subject, such as a human patient or another mammal type, to the computer system 310. In turn, the peptide assignment tool 312 processes the peptide information 322 to define peptide groups 314. The computer system 310 sends definitions of the peptide groups 314 to the user device 320 for presentation thereat.

In an example, the computer system 310 can be any suitable system that includes one or more processors and one or more memories storing computer-readable instructions executable by the one or more processors to configure the computer system 310 to host the peptide assignment tool 312 and communicate with the user device 320. For instance, the computer system 310 may be a server or a cloud computing service hosted in a data center.

In comparison, the user device 320 can be any suitable computing device that includes one or more processors and one or more memories storing computer-readable instructions executable by the one or more processors to configure the user device 320 to receive input about the peptide information 322, communicate with the computer system 310, and present the information about the peptide groups 314. For instance, the user device 320 may be a smartphone, a tablet, a laptop, a desktop computer, a server, or a cloud computing service hosted in a data center.

Although FIG. 3 illustrates the computer system 310 and the user device 320 as being two separate computing components, embodiments of the present disclosure are not limited as such. For instance, the computer system 310 and the user device 320 can be integrated as a single computing component. Further, the configuration of the user device 320 need not be limited to receiving the peptide information 322. Instead, the user device 320 can also generate the peptide information 322. For instance, the user device 322 can be implemented as a genome sequencing system that general MHC information about the subject and/or a system that hosts an artificial intelligence model that generates the peptide information 322 based on the MHC information and that, automatically or upon request, sends the peptide information 322 to the computer system 310.

In an example, the peptide information 322 is specific to the subject. For instance, the peptide information 322 identifies a set of candidate peptides (e.g., forty or some sequences of amino acids) that are found to be manufacturable and soluble and that are predicted to elicit an immunogenic response upon injection into the subject. In this illustration, the peptide information 322 can be generated by performing a biopsy (e.g., on healthy or cancerous cells of the subject) and performing genome sequencing on the biopsy (including next generation sequencing (NGS)) to determine MHC alleles of the subject. Information about the MHC alleles can be input to the artificial intelligence model that then outputs identifiers of peptides and immunogenicity response predictions per peptides including, for instance, class I and class II immunogenicity response likelihoods. Communications can occur between the user device 320 and a computing device of a manufacturer to determine manufacturability and solubility of the peptides. These communications can be automated (e.g., via web interface or application programming interfaces (APIs)) or can involve a manual process (e.g., one using electronic mail (e-mail) messaging). Thereafter, the user device 320 can rank the manufacturable and soluble peptides and send the peptide information 322 to the computer system 320. The peptide information 322 can identify each peptide, its ranking (or relative ranking with respect to the other peptides), its class I and class II immunogenicity response likelihoods, its amino acid sequence length, the number of cysteines in it (or an indication of whether more than one cysteine is included), and/or other biological and/or chemical properties of the peptide.

The information about the peptide groups 314 can include one or more vaccine plans.

Each vaccine plan can identify a number of peptide groups (e.g., four groups), each containing a subset of the peptides. The subsets can be of the same length (e.g., each identifies five peptides) and may or may not identify overlapping peptides.

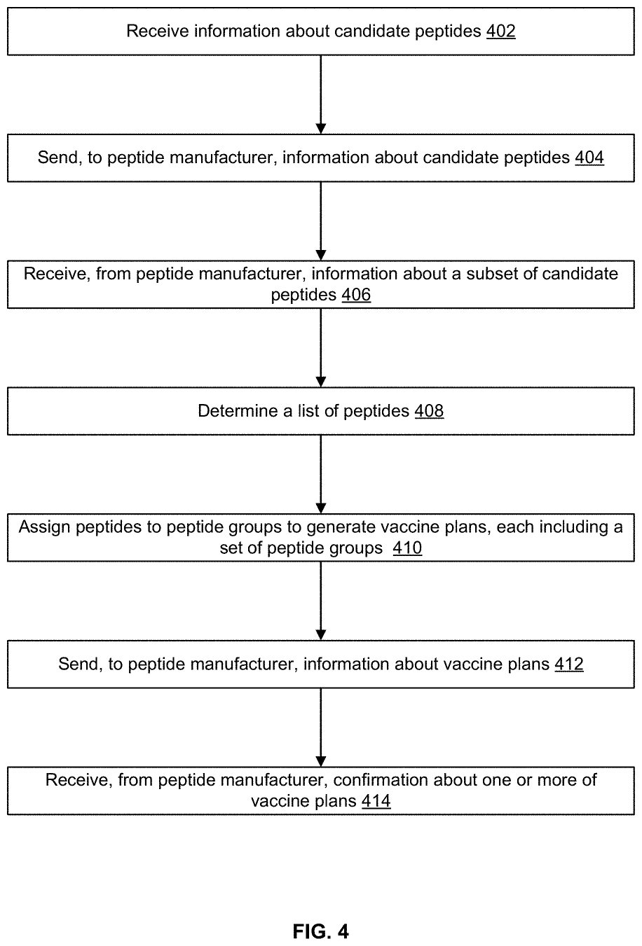

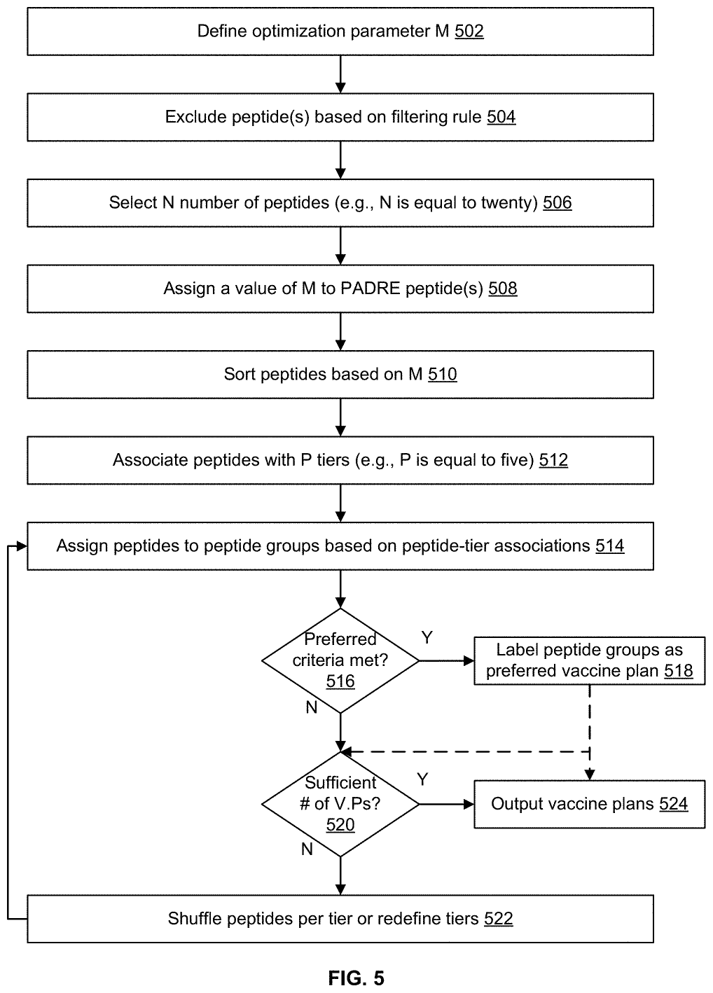

FIGS. 4-5 illustrate examples of flows for developing a cancer vaccine personalized to a subject according to embodiments of the present disclosure. A computer system and/or a user device, similar to the computer system 310 and/or user device 320 of FIG. 3, may be used to perform operations of the example flows. For example, instructions for performing the operations can be stored as computer-readable instructions on one or more non-transitory computer-readable media of the computer system and/or user device. As stored, the instructions represent programmable modules that include code or data executable by one or more processors of the computer system and/or user device. The execution of such instructions configures the computer system and/or user device to perform the specific operations shown in the corresponding figure and described herein. Each programmable module in combination with the respective processor(s) represents a means for performing a respective operation(s). While the operations are illustrated in a particular order, it should be understood that no particular order is necessary and that one or more operations may be omitted, skipped, and/or reordered. Furthermore, in the interest of clarity of explanation, various examples are provided and describe the use of twenty peptides for assignments into four peptide groups, each of identifying five of the peptides. However, the flows similarly apply to defining “P” peptide groups, with “Q” peptides from a total of “N” peptides, where “P,” “Q,” and “N” are positive integers strictly greater than one.

FIG. 4 illustrates an example of a flow for defining peptide groups for a cancer vaccine according to embodiments of the present disclosure. The peptide groups correspond to vaccine compositions or solutions, whereby peptides identified in a peptide group can be added to a solution with other ingredients to create a composition of the cancer vaccine. Other uses of the peptide groups are possible, where, for example, each peptide group can correspond to a pill composition of the cancer vaccine. Various numbers are described with the operations (e.g., sixty peptides, forty peptides, eighteen top ranked peptides, two PADRE peptides (and/or other types of universal peptides), and the like). These numbers are provide for illustrative purposes only and any set of numbers can be used.

As illustrated, the flow can start at operation 402, where information about candidate peptides are received. For example, this information identifies sixty (or some other total number) amino acid sequences predicted to trigger class I immunogenic responses and/or class II immunogenic responses in the subject. The information can include, for each neo-antigen peptide, its class I immunogenic response likelihood and/or class II immunogenic response likelihood, relative ranking, amino acid sequence, number of cysteines in the amino acid sequence, length of the amino acid sequence, and/or other biological properties and/or chemical properties of the neo-antigen peptide.

At operation 404, some or all of the information can be sent to a peptide manufacturer. For example, the information identifies at least the amino acid sequence of each neo-antigen peptide. The information can be sent, along with a request for manufacturability and solubility analysis, via a web interface, API, or a means of communications (e.g., e-mail message, file upload, and the like) to a computing device of the peptide manufacturer.

At operation 406, information about a subset of the candidate peptides is received back from the peptide manufacturer. This information can be received as a response from the peptide manufacturer's computing device and can identify at least the subset, where each neo-antigen in the subset was found to be manufacturable and soluble. For example, this subset can include forty (or some other total number) out of the sixty neo-antigen peptides.

At operation 408, a list of peptides is determined. The list includes some of the neo-antigen peptides from the subset, such as eighteen (or some other total number) out of the forty neo-antigen peptides. For example, the forty neo-antigen peptides are ranked according to one or more of the biological and/or chemical properties, including any of their class I immunogenic response likelihoods and/or class II immunogenic response likelihoods, and lengths of their amino acid sequences. The eighteen top ranked neo-antigen peptides are selected and identified in the list. Two PADRE peptides (and/or similarly other types of universal peptides) are also identified in the list for a total of twenty peptides. Of course, a different number of peptides (whether neo-antigen or peptides) can be used. In addition, multiple lists can be determined, where one or more vaccine plans can be derived from each list.

At operation 410, peptides from the list are assigned to peptide groups to generate one or more vaccine plans, each including a set of peptide groups. For example, from the list of twenty peptides, a first vaccine plan is generated and include four peptide groups, each of which identifies five of the twenty peptides. Similarly, an additional vaccine plan is generated from the same list but identifies a different assignment of the twenty peptides to four peptide groups.

Likewise, one or more vaccine plans are generated from any additional lists. The different vaccine plans can be marked with a preferred order of use (e.g., the first vaccine plan is a preferred plan relative to the additional vaccine plan). The peptide manufacturer can test the peptide co-solubility per vaccine plan according to the preferred order. When no sufficient co-solubility is found in a vaccine plan, the manufacturing moves to the next vaccine plan per the preferred order.

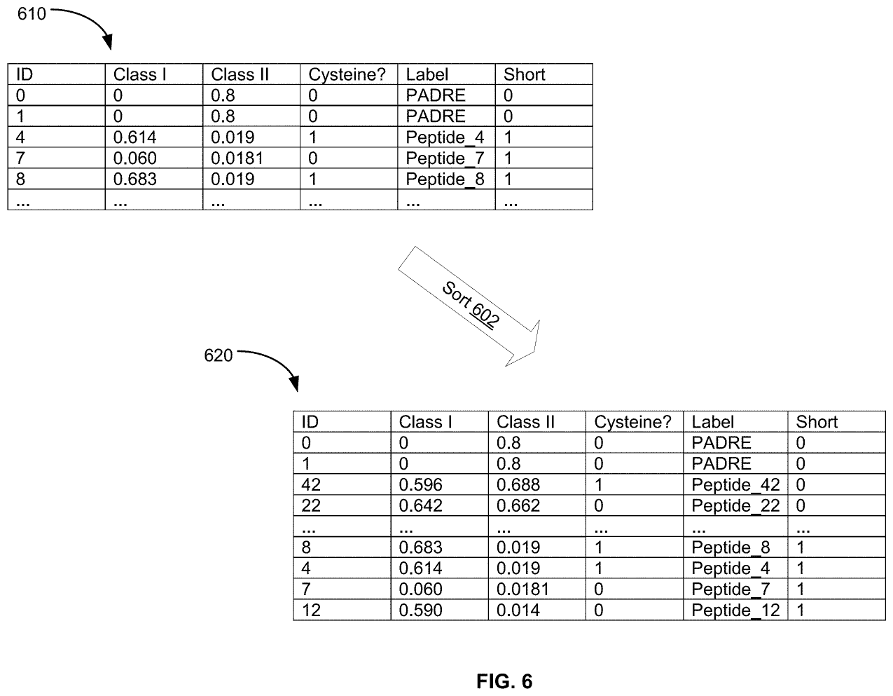

In an example, a peptide assignment tool, such as the peptide assignment tool 612 of FIG. 6, performs the peptide-to-peptide group assignment to generate the vaccine plans and define the preferred order. The assignment can be based on an optimization parameter that biases the assignment such that peptide groups in a vaccine plan have a similar group property. The optimization parameter can be any or a combination of peptide biological and/or physical properties including any of their class I immunogenic response likelihoods, class II immunogenic response likelihoods, and lengths of their amino acid sequences. A group property of a peptide group can include biological and/or chemical properties that collectively represent the individual biological and/or chemical properties of the peptide assigned to the peptide group. For example, the group property can be statistical measure (e.g., average, median, or the like) of class I immunogenic response likelihoods, class II immunogenic response likelihoods, lengths of their amino acid sequences, or a combination of such individual peptide properties. Similarity of group properties can be defined as a statistical measure (e.g., difference or standard deviation) relative to a predefined similarity range (e.g., plus/minus ten percent).

Further, the assignment can be based on assignment rules that filter out peptide groups and/or set limits on peptide duplications. For instance, a vaccine plan is removed if any of its peptide groups contains more than one peptide having a cysteine. A universal peptide, such as a PADRE peptide, may be allowed to be duplicated in no more than two (or some other limit) of the peptide groups per vaccine plan. A neo-antigen peptide may not be duplicated at all. Alternatively, depending on the neo-antigen peptide's class I or class II immunogenicity likelihood (e.g., its class I immunogenic response likelihood exceeding a predefined threshold likelihood and/or its class II immunogenic response likelihood exceeding the same or a different predefined threshold likelihood), the neo-antigen peptide may not be duplicated a number of times (e.g., up to two times such that is assigned to more than two peptide groups of the vaccine plan, or up to four times for a high scoring neo-antigen peptide such that it is assigned to each peptide group of the vaccine plan).

The peptide assignment tool can use one or more assignment algorithms to assign the twenty peptides to four peptide groups according to the optimization parameter and assignment rule(s). In a first example, an iterative random search is used. In an iteration, a random assignment is made. The group property is determined per peptide group from the individual peptide properties of the peptides that have been randomly assigned to the peptide group. The group properties are compared and, if they are within a similarity range of each other, the peptide assignment tool outputs the peptide groups as a vaccine plan. Otherwise, these peptide groups are disregarded and a next iteration is performed. Multiple iterations can also be performed to define multiple vaccine plans.

In a second example, the peptide assignment tool implements a combinatorial optimization algorithm. This algorithm can define a loss function as the similarity measure between the group properties (e.g., the difference or standard deviation). To minimize the loss function, the combinatorial optimization algorithm explores different peptide-to-peptide group assignments as variables, subject to assignment rule constraints.

In a third example, the peptide assignment tool implements a tournament style algorithm, such as the one that executes the flow of FIG. 5. Briefly, this algorithm can sort the peptides in a descending order based on one or more of their biological and/or chemical properties. Tiers can be defined and peptides can be associated thereto depending on their sorted orders. Then, peptides from the different tiers can be assigned to the peptide groups of a vaccine plan. These assignments can be subject to the assignment rules. The peptide-to-tier associations can be shuffled and/or the definitions of tiers can be updated to generate additional vaccine plans.

Upon generating multiple vaccine plans, the peptide assignment tool can mark them with a preferred order of use. This marking can depend on a number of factors. For instance, the more similar the group properties of peptide groups within a vaccine plan, the higher the vaccine plan is in the preferred order of use.

At operation 412, information about the one or more vaccine plans are sent to the peptide manufacturer. For instance, the information identifies at least, the peptide groups per vaccine plan and the preferred order of use. This information can be sent, along with a request for co-solubility analysis, via a web interface, API, or a means of communications (e.g., e-mail message, file upload, and the like) to a computing device of the peptide manufacturer.

At operation 414, a confirmation is received from the peptide manufacturer about one or of more of the vaccine plans. For instance, this confirmation is received as a response from the peptide manufacturer's computing device and indicates whether a vaccine plan includes co-soluble peptides that have been assigned to a peptide group. If all peptide groups of a vaccine plan contain co-soluble peptides, instructions can be sent to the peptide manufacturer to produce the solutions (or pills) corresponding to such peptide groups.

FIG. 5 illustrates an example of a flow for assigning peptides to peptide groups for a cancer vaccine according to embodiments of the present disclosure. This flow can represent a tournament style assignment of the peptides and can be implemented as sub-operations of operation 410 of FIG. 4. In the interest of explanation, the flow of FIG. 5 is described in connection with a specific number of peptides and specific number of peptide groups and some of its operations are further illustrated in FIGS. 6-10.

As illustrated, the flow can start at operation 502, where an optimization parameter is defined. In an example, the optimization parameter relates to reducing the likelihood for immuno-dominance. Immuno-dominance may occur because when, for instance, many neo-antigens are presented simultaneously to T-cells, there is a likelihood that the immune system will only respond to a subset but not all of them. As such, the optimization parameter can be sequence length. In particular, no peptide group (i.e., the sum of peptide lengths in a group) can be too long. The length of the peptide group can be equal to the sum of the lengths of the peptide assigned to the peptide groups. The lengths of all the peptide groups can be roughly similar (e.g., within plus/minus ten percent of each other). The length of a peptide group can be defined as the sum of the amino sequence lengths of the peptides assigned to the group. This optimization parameter makes the number of peptides with a particular length (e.g., 9-10 mer peptides) similar between peptide groups. Alternatively, the peptides can be labeled as long or short (e.g., long means longer than twenty amino acids and short means shorter than twenty amino acids) or a more granular length resolution can be used. In this case, the length of a peptide group can be defined as a certain distribution of long and short peptides (e.g., four long peptides and one short peptide). A similar distribution across the peptide groups can be aimed for in the assignment. For instance, no more than two short peptides are allowed in a peptide group. Alternatively, one peptide group may include the top ranked class I peptides (e.g., the ones with the highest class I immunogenicity likelihoods) that are short, while the remaining three peptide groups can have comparable lengths.

In another example, the optimization parameter relates to the class I immunogenicity and/or class II immunogenicity. For example, the class I immunogenicity likelihood of a peptide, as predicted for a subject by an artificial intelligence model, represents a class I score of the peptide. Likewise, the class II immunogenicity likelihood of a peptide, as predicted for a subject by an artificial intelligence model, represents a class II score of the peptide. A statistical measure (e.g., median, average, total, or the like) per peptide group can be derived from the class I scores and/or class II scores of the peptides assigned to that group. The peptide groups should have similar class I score and/or class II score (e.g., within a similarity range relative to each other).

In yet another example, the optimization parameter relates to reducing the likelihood for immuno-dominance and to the immunogenicity responses. For instance, the optimization parameter can be multi-dimension and includes, in each dimension, one of the above parameters. A dimension reduction algorithm, such a principal component analysis (PCA), can be employed to define a representative scalar for each peptide group for comparison with that of the remaining peptide groups. In the case of using PCA, a class I score, a class II score, and/or an amino sequence length per peptide is converted into a single scalar by taking the first principal component of the individual scores and projecting the neo-antigen-specific scores into the first principal component. The assignment may necessitate that the W first principal components are approximately equal amongst the pools.

Accordingly, at operation, an optimization parameter M is defined as M=Σᵢ²⁰ Mᵢ, where Mᵢ is the value of the optimization parameter for each individual peptide. For example, Mᵢ can be the class I score, the class II score, the sequence length, or the first principal component. The tournament style assignment attempts to maximize M.

At operation 504, one or more peptides are excluded based on a filtering rule. For instance, the filtering rule removes any neo-antigen peptides that meet any of the following criteria: (i) the peptide includes more than one cysteine in its sequence, (ii) the peptide cannot be synthesized by the peptide manufacturer, or (iii) the peptide is found not to be sufficiently soluble.

At operation 506, N number of peptides is selected. For instance Nis equal to twenty and includes eighteen neo-antigen peptides and two copies of the PADRE peptide (or any other type of universal peptides). Two PADRE peptide copies correspond to a duplication rule of allowing only two PADRE peptide duplicates. None of the eighteen neo-antigen peptides can be a duplicate reflecting a duplication rule prohibiting neo-antigen peptide duplicates. Alternatively, if the duplication rule permits neo-antigen peptide duplicates, some of the eighteen neo-antigen peptides can be copies of each other and the number of copies can be limited by the duplication rule. Further, such duplication rule can specify which neo-antigen peptide can be duplicated (e.g., one having an Mᵢ over a certain threshold value, such as one having a class I score or a class II score exceeding a threshold score).

At operation 508, a value of M is assigned to the PADRE peptide (the same value can be assigned to the two copies). This value can be a default value that depends on how the peptides are to be ranked such that they can be sorted in a descending order. For instance, if ranking by class II score, the PADRE peptide is assigned an M value that is better than any of the eighteen neo-antigen peptides (e.g., a value of 0.8). In this operation, and other operations of the flow, another type of PADRE peptide can be additionally or alternatively used. If so, a value of M is assigned to such universal peptide, can depend on the known immunogenicity response and/or property of the universal peptide, and is used in the remaining operations of the flow.

At operation 510, the twenty peptides are sorted based on their individual values of M (e.g., based on Mᵢ). For example, the peptides are ranked in a descending order according to their Mᵢ and are sorted in the descending order. An illustration of this sorting is shown in FIG. 6.

Referring to FIG. 6, an example of sorting 602 peptides according to embodiments of the present disclosure is illustrated. A table 610 of twenty peptides is first identified (e.g. per operation 506 of FIG. 5). The table 610 lists an identifier (D) for each peptide, its class I score (0 for the PADRE peptide copies), its class II score (default value of 0.8 for PADRE peptides), when it contains cysteine (“0” indicates it does not contain cysteine, and “1” indicates otherwise), a label of the peptide, and a classification of whether the peptide is long or short (e.g., “0” indicates short, such as being shorter than twenty amino acid long; “1” indicates long). In the table 610, neo-antigens with IDs “2” and “3” were removed per the filtering rule.

The sorting 602 is performed. In the illustration of FIG. 6, this sorting 602 uses the class II scores, although sorting on class I scores, sequence length, and/or first principal components (not shown in the table 610) are possible. The result of the sorting 602 is an updated table 620, where the peptides are identified in a descending order of their class II score. Relative to the table 610, the PADRE peptide copies stayed top ranked, whereas peptide with ID “42” was sorted to the third top spot, and so on.

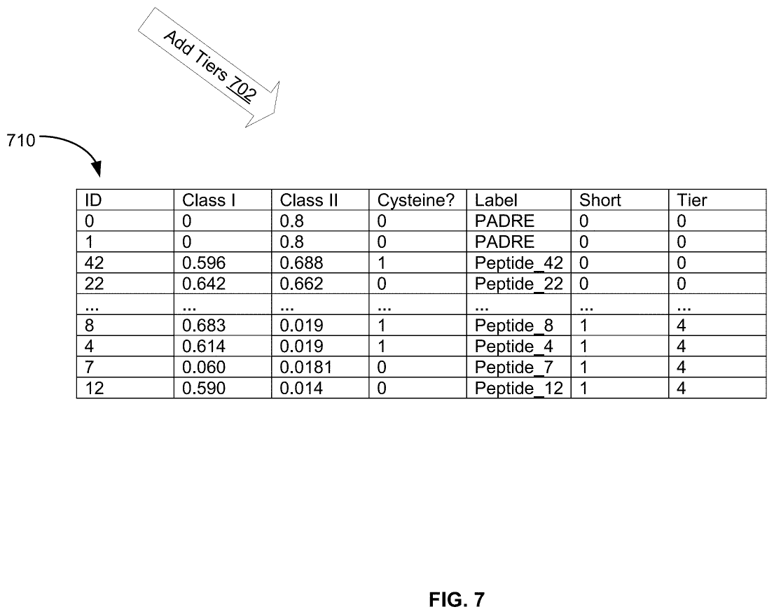

Referring back to FIG. 5, at operation 512, the sorted peptides are associated with P tiers. Generally, the number P is a positive integer equal to the number of targeted peptides per peptide group, such as five. In an example, the top four peptides are associated with tier “0,” the next four peptides are associated with tier “1,” and so on. An illustration of this tier associations is shown in FIG. 7.

Referring to FIG. 7, an example of defining tiers according to embodiments of the present disclosure is illustrated. In the illustration, table 620 of FIG. 6 is updated by adding 702 the associations of peptides with tiers to a new column of this table, resulting in an updated table 710. The added tier column identities the tier with which each peptide is associated. For instance, each of the four ranked peptides is associated with tier “0,” whereas each of the four worst ranked peptides are associated with the last tier (e.g., tier “4”).

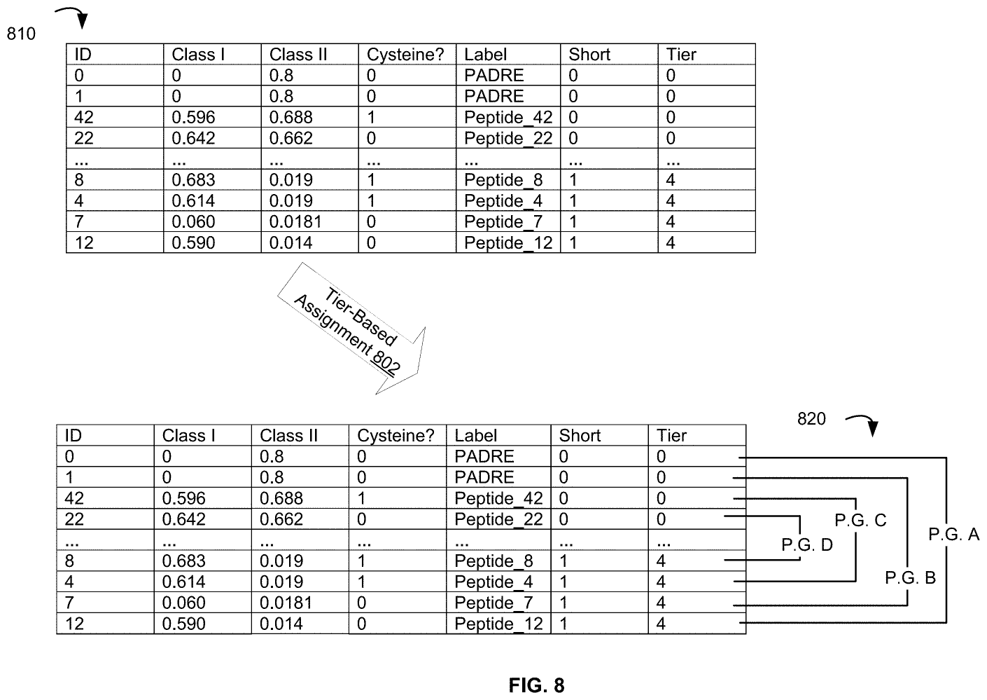

Referring back to FIG. 5, at operation 514, the peptides are assigned to peptide groups based on their associations with the tiers. For example, four peptide groups are to be defined, each to identify five of the twenty peptides. Recall that each tier is associated with four peptides. Accordingly, one peptide from each tier can be assigned to each peptide group, resulting in the four peptide groups, each having five peptides. In an example of this assignment, the top ranked peptide in the first tier (e.g., having the largest Mᵢ in this tier) and the worst ranked peptide in the last tier (e.g., having the smallest Mᵢ in this tier) can be assigned to the first peptide group. Next, the second top-ranked peptide in the first tier and the second worst-ranked peptide in the last tier can be assigned to the second peptide group, and so on until all the peptides have been assigned to the peptide groups. An illustration of this tier associations is shown in FIG. 8.

Referring to FIG. 8, an example of a tier-based assignment 802 of peptides to peptide groups according to embodiments of the present disclosure is illustrated. In the illustration, table 810 of FIG. 8 corresponds to table 720 of FIG. 7 and is used in the tier-based assignment 802. The peptides have already been ranked according to their class II scores (although a different optimization parameter M is possible). As such, the ranking described herein next refers to the class II scores. Peptide with ID “0” ranked the highest in tier “0” and is assigned to the first peptide group “A.” Peptide with ID “12” ranked the first worst in the last tier “4” and is also assigned to the first peptide group “A.” Next, peptide with ID “1” ranked the second highest in tier “0” and is assigned to the second peptide group “B.” Peptide with ID “7” ranked the second worst in the last tier “4” and is also assigned to the second peptide group “B.” Similarly, peptide with ID “42” ranked the third highest in tier “0” and is assigned to the third peptide group “C.” Peptide with ID “4” ranked the third worst in the last tier “4” and is also assigned to the third peptide group “B.” In addition, peptide with ID “22” ranked the fourth highest in tier “0” and is assigned to the fourth peptide group “D.” Peptide with ID “8” ranked the fourth worst in the last tier “4” and is also assigned to the fourth peptide group “B.” This tier-based assignment 802 is repeated in a similar fashion across the remaining tiers.

Referring back to FIG. 5, at operation 516, a determination is made as to whether one or more assignment rules are satisfied. If so, operation 518 follows operation 516. Otherwise, operation 520 follows operation 516. The assignment rule(s) can specify one or more of the following criteria for the assignment rule(s) to be satisfied: (i) no more than one or two PADRE peptides are found in the peptide groups, (ii) no neo-antigen peptide is duplicated (or, a maximum number of neo-antigen peptide duplications is met), and/or (iii) no more peptide group contains more than one peptide that contains a cysteine.

At operation 518, the peptide groups are labeled as belonging to a preferred vaccine plan. The “preferred” label can be a relative term used to indicate that, for the vaccine manufacturing, if the peptides are found to be co-soluble as assigned, this vaccine plan is to be used instead of another vaccine plan that does not carry the “preferred” label. As indicated with the dashed arrows, operation 520 can follow operation 518, or, optionally, once the preferred vaccine plan is identified, operation 524 follows operation 518.

At operation 520, a determination is made as to whether a sufficient number of vaccine plans have been defined. The smallest number is one, although a much higher number of vaccine plans can be targeted. If not, operation 522 follows operation 520. Otherwise, operation 524 follows operation 522.

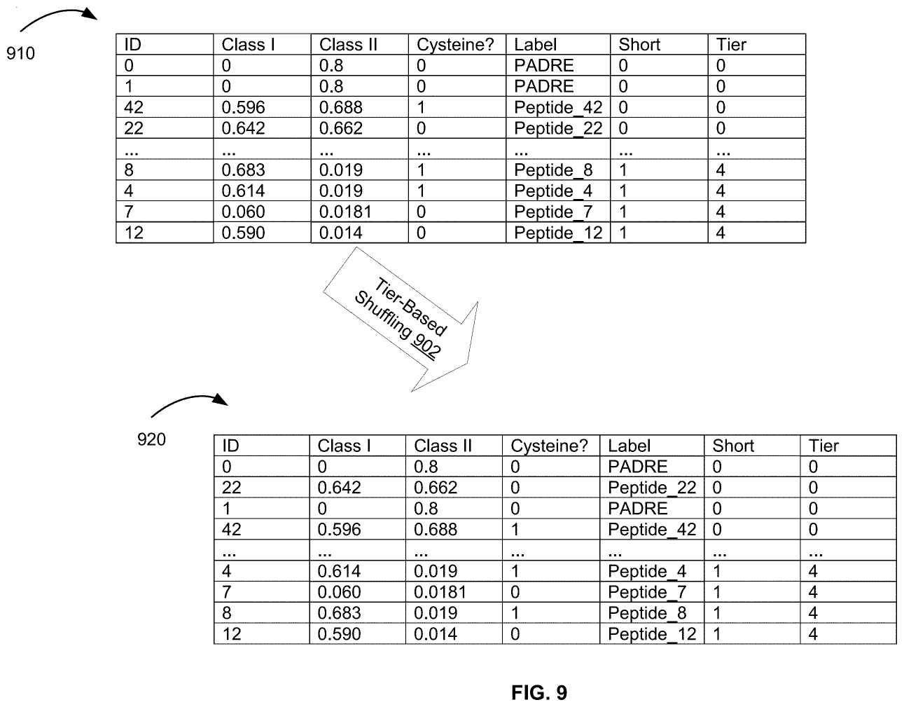

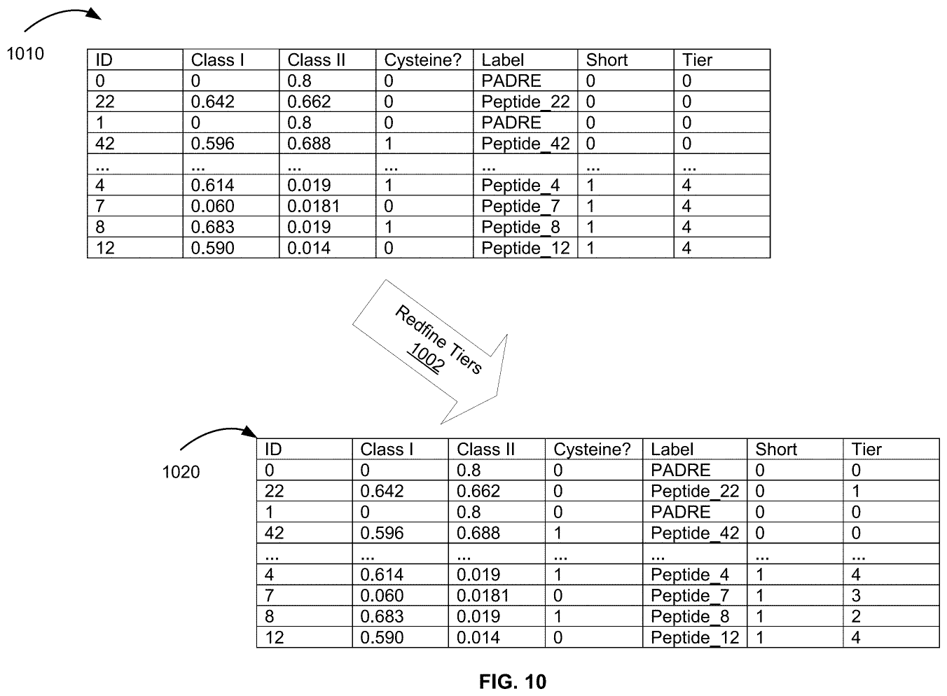

At operation 522, peptides are shuffled per tiers or the tiers are redefined. If no vaccine plan has been defined from the twenty selected peptides (per operation 506) or if an additional vaccine plan is to be defined from these peptides, the location of all peptides can be shuffled at random in a given tier, but the peptide-to-tier associations cannot be altered. An illustration of this in-tier shuffling is shown in FIG. 9. If one or more vaccine plans are to be defined from a different set of twenty peptides, the tiers can be redefined. An illustration of this tier redefinition is shown in FIG. 10. Additionally, if the tier-based shuffling of FIG. 9 does not result in a sufficient number of vaccine plans (e.g., relative to the targeted number), the tier redefinition of FIG. 10 can also be applied.

Referring to FIG. 9, an example of tier-based shuffling 902 according to embodiments of the present disclosure is illustrated. In the illustration, table 910 of FIG. 9 corresponds to table 820 of FIG. 8 and is used as the starting point of the tier-based shuffling 902. The result of the tier-based shuffling 902 is table 920.

In table 910, tier “0” is associated with peptides having IDs “0,” “1,” “42,” ad “22,” where these peptides were initially sorted in this sorting order based on their class II scores. Similarly, tier “4” is associated with peptides having IDs “8,” “4,” “7,” ad “12,” where these peptides were also initially sorted in this sorting order based on their class II scores. The tier-based assignment 902 updates the sorting order in each tier randomly and independently of their class II score. As a result, in table 920, tier “0” is still associated with peptides having IDs “0,” “1,” “42,” ad “22.” However, these peptides are now sorted randomly, where the updated sorting order lists the peptides in the descending order of IDs “0,” “22,” “1”, and “42”. Also in table 920, tier “4” is still associated with peptides having IDs “8,” “4,” “7,” ad “12.” However, these peptides are now sorted randomly, where the updated sorting order lists the peptides in the descending order of IDs “4,” “7,” “8”, and “12.” Similar random shuffling can be performed in each of the tiers.

Referring to FIG. 10, an example of redefining tiers 1002 according to embodiments of the present disclosure is illustrated. In the illustration, table 1010 of FIG. 10 corresponds to table 920 of FIG. 9 or to a new table in which one or more peptides have been replaced by one or more peptides that were not selected at operation 506 (e.g., a neo-antigen peptide was replaced by another one). The table 1010 is used as the starting point of the tier re-definition, the result of which is table 1020.

In an example, two new super-tiers can be defined based on whether a peptide is below or above the median (or some other statistical measure) of the class II score. The peptides in each super-tiers are shuffled randomly, resulting in table 1020. For instance, whereas peptide with ID “0” remains in tier “0” after this reshuffling, the association of peptide with ID “22” changes from tier “0” to tier “1.”

Referring back to FIG. 5, a loop exists from operation 522 to operation 514. In this way, after the tier-based shuffling 902 of FIG. 9 and/or the tier re-redefinition of FIG. 10, new peptide-to-peptide group assignments can be identified.

At operation 524, the vaccine plans are output. For instance, each vaccine plan identifies the peptide-to-peptide group assignment and whether it is a preferred plan or not.

In the above illustrations, a neo-antigen peptide may have been predicted as having a high class I score and/or a high class II score (e.g., by comparing these scores to corresponding threshold score(s)). If so, a neo-antigen peptide may allow duplication of this neo-antigen in two or more of the peptide groups. Additionally or alternatively, the subject may have a localized tumor (e.g., one growing in one of the four quadrants described in FIG. 1). Because this neo-antigen peptide is predicted to have a high immunogenicity response, the vaccine plan can associate the peptide group to which this peptide is assigned with the quadrant (or location) where the resulting vaccine shot is to be injected. For instance, if the tumor is growing in the quadrant “Q1,” the vaccine plan can indicate that a vaccine shot that includes this high efficacy peptide is to be injected in the top left arm.

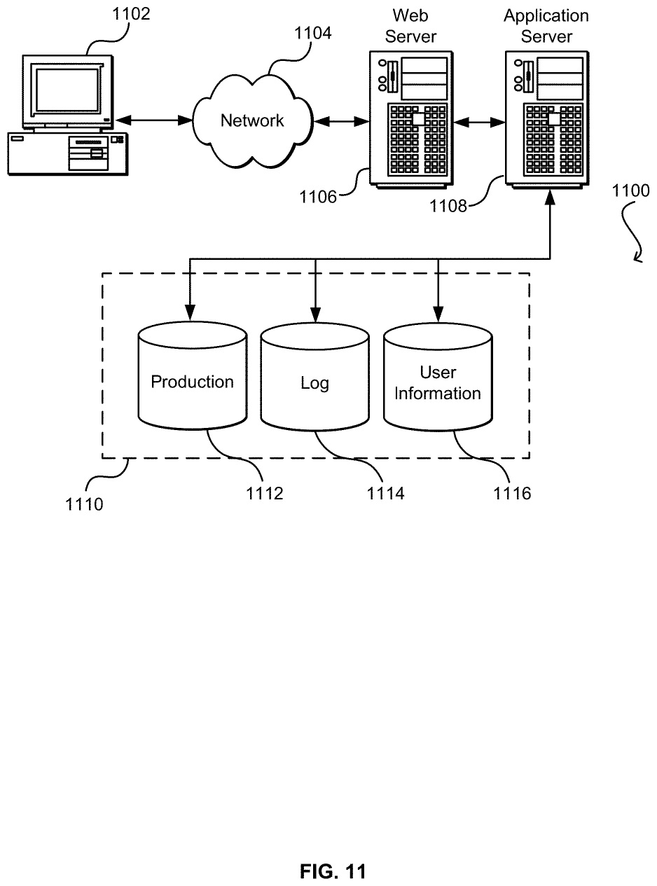

FIG. 11 illustrates aspects of an example environment for implementing aspects in accordance with various embodiments. This architecture may be used to implement some or all of the components of the computer systems (e.g., the computer system 310 of FIG. 3) described herein above. The computer architecture shown in FIG. 11 illustrates a server computer, workstation, desktop computer, laptop, tablet, network appliance, personal digital assistant (“PDA”), e-reader, digital cellular phone, or other computing device, and may be utilized to execute any aspects of the software components presented herein.

The computer 1100 includes a baseboard 1102, or “motherboard,” which is a printed circuit board to which a multitude of components or devices may be connected by way of a system bus or other electrical communication paths. In one illustrative embodiment, one or more central processing units (“CPUs”) 1104 operate in conjunction with a chipset 1106. The CPUs 1104 may be standard programmable processors that perform arithmetic and logical operations necessary for the operation of the computer 1100.

The CPUs 1104 perform operations by transitioning from one discrete, physical state to the next through the manipulation of switching elements that differentiate between and change these states. Switching elements may generally include electronic circuits that maintain one of two binary states, such as flip-flops, and electronic circuits that provide an output state based on the logical combination of the states of one or more other switching elements, such as logic gates. These basic switching elements may be combined to create more complex logic circuits, including registers, adders-subtractors, arithmetic logic units, floating-point units, and the like.

The chipset 1106 provides an interface between the CPUs 1104 and the remainder of the components and devices on the baseboard 1102. The chipset 1106 may provide an interface to a random access memory (“RAM”) 1108, used as the main memory in the computer 1100. The chipset 1106 may further provide an interface to a computer-readable storage medium such as a read-only memory (“ROM”) 1110 or non-volatile RAM (“NVRAM”) for storing basic routines that help to startup the computer 1100 and to transfer information between the various components and devices. The ROM 1110 or NVRAM may also store other software components necessary for the operation of the computer 1100 in accordance with the embodiments described herein.

The computer 1100 may operate in a networked environment using logical connections to remote computing devices and computer systems through a network, such as the local area network 1120. The chipset 1106 may include functionality for providing network connectivity through a NIC 1112, such as a gigabit Ethernet adapter. The NIC 1112 is capable of connecting the computer 1100 to other computing devices over the network 1120. It should be appreciated that multiple NICs 1112 may be present in the computer 1100, connecting the computer to other types of networks and remote computer systems.

The computer 1100 may be connected to a mass storage device 1118 that provides non-volatile storage for the computer. The mass storage device 1118 may store system programs, application programs, other program modules, and data, which have been described in greater detail herein. The mass storage device 1118 may be connected to the computer 1100 through a storage controller 1114 connected to the chipset 1106. The mass storage device 1118 may consist of one or more physical storage units. The storage controller 1114 may interface with the physical storage units through a serial attached SCSI (“SAS”) interface, a serial advanced technology attachment (“SATA”) interface, a fiber channel (“FC”) interface, or other type of interface for physically connecting and transferring data between computers and physical storage units.

The computer 1100 may store data on the mass storage device 1118 by transforming the physical state of the physical storage units to reflect the information being stored. The specific transformation of physical state may depend on various factors, in different implementations of this description. Examples of such factors may include, but are not limited to, the technology used to implement the physical storage units, whether the mass storage device 1118 is characterized as primary or secondary storage, and the like.

For example, the computer 1100 may store information to the mass storage device 1118 by issuing instructions through the storage controller 1114 to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computer 1100 may further read information from the mass storage device 1118 by detecting the physical states or characteristics of one or more particular locations within the physical storage units.

In addition to the mass storage device 1118 described above, the computer 1100 may have access to other computer-readable storage media to store and retrieve information, such as program modules, data structures, or other data. It should be appreciated by those skilled in the art that computer-readable storage media can be any available media that provides for the storage of non-transitory data and that may be accessed by the computer 1100.

By way of example, and not limitation, computer-readable storage media may include volatile and non-volatile, removable and non-removable media implemented in any method or technology. Computer-readable storage media includes, but is not limited to, RAM, ROM, erasable programmable ROM (“EPROM”), electrically-erasable programmable ROM (“EEPROM”), flash memory or other solid-state memory technology, compact disc ROM (“CD-ROM”), digital versatile disk (“DVD”), high definition DVD (“HD-DVD”), BLU-RAY, or other optical storage, magnetic cassettes, magnetic tape, magnetic disk storage or other magnetic storage devices, or any other medium that can be used to store the desired information in a non-transitory fashion.

The mass storage device 1118 may store an operating system 1130 utilized to control the operation of the computer 1100. According to one embodiment, the operating system comprises the LINUX operating system. According to another embodiment, the operating system comprises the WINDOWS® SERVER operating system from MICROSOFT Corporation.

According to further embodiments, the operating system may comprise the UNIX or SOLARIS operating systems. It should be appreciated that other operating systems may also be utilized. The mass storage device 1118 may store other system or application programs and data utilized by the computer 1100. The mass storage device 1118 might also store other programs and data not specifically identified herein.

In one embodiment, the mass storage device 1118 or other computer-readable storage media is encoded with computer-executable instructions which, when loaded into the computer 1100, transforms the computer from a general-purpose computing system into a special-purpose computer capable of implementing the embodiments described herein. These computer-executable instructions transform the computer 1100 by specifying how the CPUs 1104 transition between states, as described above. According to one embodiment, the computer 1100 has access to computer-readable storage media storing computer-executable instructions which, when executed by the computer 1100, perform the various routines described above. The computer 1100 might also include computer-readable storage media for performing any of the other computer-implemented operations described herein.

The computer 1100 may also include one or more input/output controllers 1116 for receiving and processing input from a number of input devices, such as a keyboard, a mouse, a touchpad, a touch screen, an electronic stylus, or other type of input device. Similarly, the input/output controller 1116 may provide output to a display, such as a computer monitor, a flat-panel display, a digital projector, a printer, a plotter, or other type of output device. It will be appreciated that the computer 1100 may not include all of the components shown in FIG. 11, may include other components that are not explicitly shown in FIG. 11, or may utilize an architecture completely different than that shown in FIG. 11. It should also be appreciated that many computers, such as the computer 1100, might be utilized in combination to embody aspects of the various technologies disclosed herein.

Also provided are vaccines, for example vaccines comprising a plurality of different vaccine compositions, wherein each vaccine composition includes a different set of peptides from a plurality of peptides predicted to cause an immunogenicity response in a subject, wherein each vaccine composition has a property that is within a similarity range of properties of the remaining vaccine compositions (for example as described above), wherein the property comprises at least one of a class I immunogenicity score, a class II immunogenicity score, or amino acid sequence lengths. Each vaccine can be in a different container or can be other separated from each other, for example in different ampules or vials.

Also provided are vaccines comprising a plurality of different vaccine compositions identified as described herein. For example, each vaccine composition can comprise a different set of peptides identified from a plurality of different groups of peptides, wherein the plurality of different groups are defined by at least: determining a peptide property of a peptide from different peptides that are to be assigned to the plurality of different groups, the different peptides predicted to generate an immunogenicity response in a subject; determining that the peptide is to be assigned to a first group from the plurality of different groups based at least in part on the peptide property, the first group having a first group property that is based at least in part on peptide properties of first peptides to be assigned to the first group, the first group property being within a similarity range relative to a second group property of a second group from the plurality of different groups; and generating information indicating that the peptide is assigned to the first group.

Also provided are methods of inducing an immune response in a subject (e.g., a human, other mammal or other animal, for example such as a bird) with the vaccines described herein.

In some embodiments, the method can comprise administering to the subject one or more vaccine compositions of peptides from a plurality of vaccine compositions (e.g., as described above), thereby inducing an immune response to one or more peptides in the one or more vaccine compositions.

In some embodiments, the vaccine is a cancer vaccine and the plurality of vaccine compositions are generated from information that assigns neo-antigen peptides predicted to generate an immunogenicity response in subject to a plurality of groups of peptides, wherein a first group of peptides of the plurality of groups has a first group property being within a similarity range relative to a second group property of a second group of peptides of the plurality of groups. In some embodiments, two or more vaccine compositions from the plurality are administered to the subject. In some embodiments, the subject is the single subject for which neo-antigen peptides were predicted to predict an immunogenicity response and are assigned to the plurality of groups.

The vaccines can be formulated using a sufficient amount of each peptide to generate an immune response. In some embodiments, the vaccines are formulated to contain a final concentration of each peptide in the range of from 0.2 to 200 μg/ml, e.g., 5 to 50 μg/ml. It will be appreciated that other concentrations may also be used and each peptide can be assayed separately or together to determine optimal concentration(s) for inducing an immune response.

The peptides can be modified to alter, for example, their in vivo stability. For instance, inclusion of one or more D-amino acids in the peptide typically increases stability, particularly if the D-amino acid residues are substituted at one or both termini of the peptide sequence or the peptide can be PEGylated, for example. Stability can be assayed in a variety of ways such as by measuring the half-life of the proteins during incubation with peptidases or human plasma or serum. A number of such protein stability assays have been described (see, e.g., Verhoef et al., Eur. J. Drug Metab. Pharmacokin. 11:291-302 (1986).

A vaccine or vaccine composition can be prepared in some embodiments as an injectable, either as a liquid solution or suspension. Injection may be subcutaneous, intramuscular, intravenous, intraperitoneal, intrathecal, intradermal, intraepidermal, or by “gene gun”. Other types of administration comprise electroporation, implantation, suppositories, oral ingestion, enteric application, inhalation, aerosolization or nasal spray or drops. Solid forms, suitable for dissolving in, or suspension in, liquid vehicles prior to injection may also be prepared. The preparation may also be emulsified or encapsulated in liposomes for enhancing adjuvant effect.

A liquid formulation may include, for example, oils, polymers, vitamins, carbohydrates, amino acids, salts, buffers, albumin, surfactants, or bulking agents. Exemplary carbohydrates include sugar or sugar alcohols such as mono-, di-, or polysaccharides, or water-soluble glucans. The saccharides or glucans can include for example fructose, dextrose, lactose, glucose, mannose, sorbose, xylose, maltose, sucrose, dextran, pullulan, dextrin, alpha and beta cyclodextrin, soluble starch, hydroxyethyl starch and carboxymethylcellulose, or mixtures thereof. “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to the amount used as long as the sugar or sugar alcohol is soluble in the aqueous preparation. In some embodiments, the sugar or sugar alcohol concentration is between 1.0% (w/v) and 7.0% (w/v), e.g., between 2.0 and 6.0% (w/v). Exemplary amino acids include levorotary (L) forms of carnitine, arginine, and betaine; however, other amino acids may be added. Exemplary polymers include polyvinylpyrrolidone (PVP) with an average molecular weight between 2,000 and 3,000, or polyethylene glycol (PEG) with an average molecular weight between 3,000 and 5,000. In some embodiments, one can use a buffer in the composition to minimize pH changes in the solution before lyophilization or after reconstitution. Any physiological buffer may be used, but in some cases can be selected form citrate, phosphate, succinate, and glutamate buffers or mixtures thereof.

The terms “polypeptide,” “peptide” and “protein” are used interchangeably herein to refer to a polymer of amino acid residues. The terms encompass to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The specification and drawings are, accordingly, to be regarded in an illustrative rather than a restrictive sense. It will, however, be evident that various modifications and changes may be made thereunto without departing from the broader spirit and scope of the disclosure as set forth in the claims.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions and equivalents falling within the spirit and scope of the invention, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this disclosure are described herein, including the best mode known to the inventors for carrying out the invention. Variations of those preferred embodiments may become apparent to those of ordinary skill in the art upon reading the foregoing description. The inventors expect skilled artisans to employ such variations as appropriate and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications and patents, cited herein are hereby incorporated by reference to the same extent as if each reference were individually and specifically indicated to be incorporated by reference and were set forth in its entirety herein.

What is claimed is:

1. A system, comprising:
one or more processors; and
one or more memories storing computer-readable instructions that, upon execution by the one or more processors, configure the system to:
determine, for a subject, different peptides to be assigned to different groups of cancer vaccine for the subject, wherein each group comprises two or more different peptides;
determine a peptide property of a peptide from the different peptides, the peptide property comprising at least one of: a class I immunogenicity score, a class II immunogenicity score, or an amino acid sequence length;
define a first group of the different groups by assigning first peptides from the different peptides to the first group, wherein:
the first peptides are assigned to the first group based at least in part on the peptide property;
the first group has a first group property that comprises a measure of at least one of: class I immunogenicity scores, class II immunogenicity scores, or amino acid sequence lengths of the first peptides; and
the first group property is within a similarity range relative to a second group property of a second group from the different groups; and
generate information indicating that the first peptides are assigned to the first group.

2. The system of claim 1, wherein the one or more memories store further computer-readable instructions that, upon execution by the one or more processors, configure the system to:
determine a sorted order of the different peptides based at least in part on individual peptide properties;
associate, based at least in part on the sorted order, a first subset of the different peptides with a first tier and a second subset of the different peptides with a second tier, the peptide being a first peptide associated to the first tier; and
assign, to the first group, the first peptide associated with the first tier and a second peptide associated with the second tier.

3. The system of claim 2, wherein the sorted order indicates that the first peptide has the top-ranked peptide property and the second peptide has the worst-ranked peptide property.

4. The system of claim 1, wherein the one or more memories store further computer-readable instructions that, upon execution by the one or more processors, configure the system to:
execute a combinatorial optimization algorithm configured to (i) determine potential assignments of the different peptides to the different groups, (ii) compute, for each group of the different groups, a group property based at least in part peptide properties of peptides potentially assigned to the group, and (iii) reduce a difference between group properties of the different groups.

5. A method, comprising:
determining a peptide property of a peptide from different peptides that are to be assigned to different groups of vaccine;
determining that the peptide is to be assigned to a first group from the different groups based at least in part on the peptide property, the first group having a first group property that is based at least in part on peptide properties of first peptides to be assigned to the first group, the first group property being within a similarity range relative to a second group property of a second group from the different groups; and
generating information indicating that the peptide is assigned to the first group.

6. The method of claim 5, further comprising:
determining that the different peptides are associated with a subject, wherein the different groups are assigned a same number of peptides and are defined for a cancer vaccine of the subject.

7. The method of claim 5, further comprising:
determining that the peptide is also assigned to the second group; and
removing the second group from a candidate set of groups of vaccine.

8. The method of claim 5, further comprising:
determining that the second group is assigned more than one peptide having a particular amino acid; and
removing the second group from a candidate set of groups of vaccine.

9. The method of claim 5, further comprising:
defining the different groups by assigning the different peptides to the different groups, wherein only a subset of the different groups is assigned PADRE peptides, and wherein no more than one PADRE peptide is assigned per group of the subset.

10. The method of claim 5, further comprising:
defining the different groups by assigning the different peptides to the different groups, wherein the different peptides comprise a neo-antigen peptide, and wherein the neo-antigen peptide is assigned to only one of the different groups.

11. The method of claim 5, further comprising:
determining that the peptide is a neo-antigen peptide that has a peptide property score larger than a threshold score, wherein the peptide property score comprises at least one of: a class I immunogenic response score or a class II immunogenic response score; and
defining the different groups by assigning the different peptides to the different groups, wherein the neo-antigen peptide is assigned to more than one group based at least in part on the peptide property score being larger than the threshold score.

12. The method of claim 5, further comprising:
determining that the different peptides are associated with a subject that has a tumor in an area;
determining that the peptide is a neo-antigen peptide that has a peptide property score larger than a threshold score; and
associating the first group with the area based at least in part on the neo-antigen peptide being assigned to the first group.

13. One or more non-transitory computer-readable storage media storing instructions that, upon execution on a system, cause the system to perform operations comprising:
determining a peptide property of a peptide from different peptides that are to be assigned to different groups of vaccine;
determining that the peptide is to be assigned to a first group from the different groups based at least in part on the peptide property, the first group having a first group property that is based at least in part on peptide properties of first peptides to be assigned to the first group, the first group property being within a similarity range relative to a second group property of a second group from the different groups; and
generating information indicating that the peptide is assigned to the first group.

14. The one more non-transitory computer-readable storage media of claim 13, further storing additional instructions that, upon execution on the system, cause the system to perform operations comprising:
defining the different groups by assigning the different peptides to the different groups, wherein the different groups are assigned a same number of peptides.

15. The one more non-transitory computer-readable storage media of claim 13, further storing additional instructions that, upon execution on the system, cause the system to perform operations comprising:
determining a sorted order of the different peptides based at least in part on individual peptide properties;
associating, based at least in part on the sorted order, a first subset of the different peptides with a first tier and a second subset of the different peptides with a second tier, the peptide being a first peptide associated to the first tier; and
assigning, to the first group, the first peptide associated with the first tier and a second peptide associated with the second tier.

16. The one more non-transitory computer-readable storage media of claim 15, wherein the second tier is associated with second peptides having a second sorted order, and wherein one more non-transitory computer-readable storage media store further instructions that, upon execution on the system, cause the system to perform operations comprising:
determining an updated order of the second subset by shuffling the second sorted order;
defining an updated first group based at least in part on the updated order; and
associating the first group with a first vaccine plan and the updated first group with a second vaccine plan.

17. The one more non-transitory computer-readable storage media of claim 16,
determining that each group associated with the first vaccine plan is not assigned more than one peptide having a particular amino acid; and
generating information indicating that the first vaccine plan is preferred relative to the second vaccine plan.

18. The one more non-transitory computer-readable storage media of claim 15, wherein the first tier and the second tier are sorted in a second sorted order, and wherein one more non-transitory computer-readable storage media store further instructions that, upon execution on the system, cause the system to perform operations comprising:
determining an updated order of the first tier and the second tier by shuffling the second sorted order; and
defining an updated first group based at least in part on the updated order.

19. The one more non-transitory computer-readable storage media of claim 13, further storing additional instructions that, upon execution on the system, cause the system to perform operations comprising:
determining a total number of peptides to assign to the different groups;
generating a peptide set by associating the peptide with the peptide set and dissociating a second peptide from the different peptides with the peptide set, wherein a size of the peptide set is equal to the total number;
defining the different groups by assigning subsets of the peptide set to the different groups;
generating an updated peptide set by disassociating the peptide with the peptide set and associating the second peptide with the peptide set, wherein a size of the updated peptide set is equal to the total number; and
defining additional groups by assigning subsets of the updated peptide set to the additional groups.

20. The one more non-transitory computer-readable storage media of claim 13, further storing additional instructions that, upon execution on the system, cause the system to perform operations comprising:
executing a combinatorial optimization algorithm configured to (i) determine potential assignments of the different peptides to the different groups, (ii) compute, for each group of the different groups, a group property based at least in part peptide properties of peptides potentially assigned to the group, and (iii) and reduce a difference between group properties of the different groups.

21. A vaccine comprising a plurality of different vaccine compositions, wherein each vaccine composition corresponds to a different group of peptides from a plurality of different groups of peptides, wherein the plurality of different groups are defined by at least:
determining a peptide property of a peptide from different peptides that are to be assigned to the plurality of different groups, the different peptides predicted to generate an immunogenicity response in a subject;
determining that the peptide is to be assigned to a first group from the plurality of different groups based at least in part on the peptide property, the first group having a first group property that is based at least in part on peptide properties of first peptides to be assigned to the first group, the first group property being within a similarity range relative to a second group property of a second group from the plurality of different groups; and
generating information indicating that the peptide is assigned to the first group.

22. The vaccine peptides of claim 21, wherein the first group property is within the similarity range to the second group property based at least in part on a comparison of class I immunogenicity scores, class II immunogenicity scores, or amino acid sequence lengths of the peptides included in the first group and the second group.

23. A method of inducing an immune response in a subject, the method comprising administering to the subject one or more vaccine compositions of peptides from a plurality of vaccine compositions, thereby inducing an immune response to one or more peptides in the one or more vaccine compositions, wherein the plurality of vaccine compositions are generated from information that assigns neo-antigen peptides predicted to generate an immunogenicity response in subject to a plurality of groups of peptides, wherein a first group of peptides of the plurality of groups has a first group property being within a similarity range relative to a second group property of a second group of peptides of the plurality of groups.

24. The method of claim 23, wherein two or more groups of vaccine compositions from the plurality are administered to the subject.

25. The method of claim 23, wherein the subject is the single subject for which neo-antigen peptides were predicted to predict an immunogenicity response and are assigned to the plurality of groups.

26. A vaccine comprising a plurality of different vaccine compositions, wherein each vaccine composition includes a different set of peptides from a plurality of peptides predicted to cause an immunogenicity response in a subject, wherein each vaccine composition has a property that is within a similarity range of properties of the remaining vaccine compositions, wherein the property comprises at least one of a class I immunogenicity score, a class II immunogenicity score, or amino acid sequence lengths.
